(19) United States
(12) Patent Application Publication
(10) Pub. No.: US 2010/0184987 A1
(43) Pub. Date: Jul. 22, 2010

(54) Preparation of Retapamulin via its Pleuromutilin-thiol precursor

(75) Inventors: Nimer Jaber, Abu-Ghosh (IL); Lilach Hedvati, Ein-Shemer (IL); Eyal Gilboa, Bat-Yam (IL); Sharon Avhar-Maydan, Givataym (IL)

Correspondence Address: KENYON & KENYON LLP, ONE BROADWAY, NEW YORK, NY, 10004, US

(73) Assignee: Teva Pharmaceutical Industries Ltd.

(21) Appl. No.: 12/617,465

(22) Filed: Nov. 12, 2009

Related U.S. Application Data
(60) Provisional application No. 61/114,361, filed on Nov. 13, 2008.
(60) Provisional application No. 61/150,121, filed on Feb. 5, 2009.

(51) Int. Cl. C07D 451/04; C07C 323/22

(52) U.S. Cl. 546/128; 560/147

(57) ABSTRACT

Provided are processes for preparation of Retapamulin via its pleuromutilin-thiol precursor.

### CROSS-REFERENCE

The present invention claims the benefit of U.S. Provisional Application No. 61/114,361 filed Nov. 13, 2008, and U.S. Provisional Application No. 61/150,121 filed Feb. 5, 2009, both of which are hereby incorporated by reference.

### FIELD OF THE INVENTION

The invention encompasses processes for preparing Retapamulin intermediates and the preparation of Retapamulin thereof.

### BACKGROUND OF THE INVENTION

Retapamulin [CAS number: 224452-66-8] has the chemical name 5-Acetic acid, [[(3-exo)-8-methyl-8-azabicyclo[3.2.1]oct-3-yl]thio]-,(3aS,4R,5S,6S,8R,9R,9aR,10R)-6-ethenyldecahydro-5-hydroxy-4,6,9,10-tetramethyl-1-oxo-3a,9-propano-3aH-cyclopentacycloocten-8-yl ester and the following chemical structure:

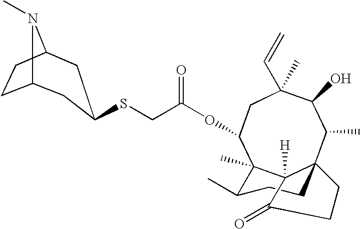

Retapamulin is disclosed in U.S. Pat. No. 6,281,226 and U.S. RE39128. Retapamulin is used in the treatment of secondarily-infected traumatic lesions (“SITL”). Processes for preparation of Retapmulin are disclosed in International Publication No. WO 99/21855 and U.S. Publication No. 2006/0276503. Polymorphic form of Retapamulin has been disclosed in International Publication No. W02006/092334. Retapamulin is also disclosed in US2009234125, WO2009095634, WO2009075776, and US2009076071, incorporated herein by reference.

### SUMMARY OF THE INVENTION

In a first embodiment, the present invention encompasses a process for preparing a pleuromutilin (PLM) derivative of Formula III (PLM-thiol) comprising: combining Formula II (PLM-OLG), an organic solvent, optionally in mixture with water, S-donor and a base to obtain a reaction mixture; and maintaining the reaction mixture to obtain the pleuromutilin (PLM) derivative of Formula III (PLM-thiol).

Optionally, When the S-donor is thiourea, PLM-thiol precursor of Formula A (PLM-thiourea ester) is obtained, and further converted to PLM-thiol.

Optionally, when the S-donor is thioacetatic acid, PLM-thiol precursor, pleoromutilin-thioacetate ester, of Formula A′ (PLM-SAc) is obtained, and further converted to PLM-thiol.

In another embodiment, the invention encompasses a process for preparing Retapamulin comprising: combining PLM-thiol, organic solvent, base and tropine-OLG to obtain a reaction mixture; and maintaining the reaction mixture to obtain the Retapamulin.

In another embodiment the present invention encompasses a process for preparing Retapamulin comprising: combining PLM-OLG, an organic solvent, optionally in mixture with water, S-donor and a base to obtain a reaction mixture; maintaining the reaction mixture to obtain the pleuromutilin derivative of PLM-thiol; further combining PLM-thiol, organic solvent, base and tropine-OLG; and maintaining the reaction mixture to obtain the Retapamulin.

In one embodiment, the invention encompasses a one pot reaction for obtaining Retapamulin comprising: converting PLM-OLG or combining PLM-thiol precursor to PLM-thiol; combining the PLM-thiol with tropine-OLG, base, and organic solvent to obtain a reaction mixture; and maintaining the reaction mixture to obtain the Retapamulin.

In another embodiment, the present invention provides a one pot reaction for obtaining Retapamulin comprising:
a) converting Tropine to Tropine-OLG in the presence of a first solvent selected from the group consisting of: acetone, MIBK and THF;
b) combining Tropine-OLG with a second solvent;
c) combining the mixture of Tropine-OLG and the second solvent with PLM-thiol precursor or PLM-thiol and a base to obtain a reaction mixture; and
d) maintaining the reaction mixture to obtain the Retapamulin.

In one embodiment, the present invention provides a one pot process for the preparation of Retapamulin comprising:

a) in a first vessel, converting PLM to PLM-OLG and further converting the PLM-OLG to PLM-thiol precursor or to PLM-thiol;

b) in a second vessel, converting Tropine to Tropine-OLG in a first solvent that is acetone;

c) combining the reaction mixture of step b) with a second solvent;

d) combining the PLM-thiol precursor or PLM-thiol with tropine-OLG, and a base to obtain a reaction mixture; and

e) maintaining the reaction mixture to obtain the Retapamulin.

### DETAILED DESCRIPTION OF THE INVENTION

As used herein, “one pot reaction” refers to a reaction which includes two or more sequential reactions without the isolation of the intermediates.

As used herein, the term “room temperature” refers to a temperature of about 20° C. to about 35° C., more preferably about 20° C. to about 25° C. and most preferably about 25° C.

As used herein, the term “sufficient time” refers to a period of time of about 2 hours to about 50 hours.

As used herein, the term “over night’ refers to a period of time of about 6 hours to about 24 hours; more preferably, about 10 hours to about 20 hours; even more preferably, about 14 hours to about 18 hours; and most preferably, about 16 hours.

In one embodiment, the present invention encompasses a process for preparing a pleuromutilin derivative of the following Formula III (PLM-thiol)

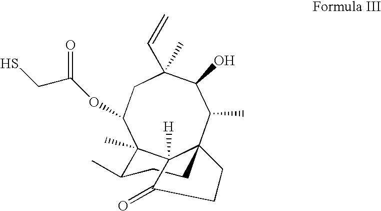

comprising: combining Formula II (PLM-OLG), an organic solvent, optionally in mixture with water, S-donor and a base to obtain a reaction mixture; and maintaining the reaction mixture to obtain PLM-thiol. Preferably, the reaction mixture is maintained for about 2 hours to about 48 hours; more preferably, for about 2 hours to about 36 hours.

Preferably, the reaction mixture is maintained at a temperature of about 0° C. to about reflux to facilitate the formation of PLM-thiol. More preferably, the reaction mixture is maintained at a temperature of about room temperature to about reflux.

The organic solvent used in the reaction includes but not limited to: C₆-C₉ aromatic aromatic hydrocarbons, C₁-C₈ alcohol, C₃-C₈ ketone, C₃-C₆ ester and C₂-C₈ ethers. More preferably, the solvent includes but not limited to: ethanol, THF, MIBK, acetone, EtOAc, toluene, methyl tert-butyl ether (MTBE) and mixtures thereof.

The base used in the reaction includes but not limited to: sodium thio-sulfite, amines, poly ethylene amines, and alkaline hydroxides. Preferably, the base is sodium metabisulfite or ethylenediamine.

The S-donor used in the reaction includes but not limited to: thiourea, thioacetic acid and salts thereof, sodium sulfide, sodium hydrosulfide, sodium xanthate. Preferably, the S-donor is thiourea or thioacetic acid.

Optionally, when the S-donor is thiourea, PLM-thiol precursor of Formula A (PLM-thiourea ester) is obtained, and further converted to PLM-thiol.

Optionally, when the S-donor is thioacetatic acid or a salt thereof, PLM-thiol precursor is pleoromutilin-thioacetate ester, of Formula A′ (PLM-SAc) is obtained, and further converted to PLM-thiol.

PLM-thiol can be recovered by combining the reaction mixture with a water immsicible solvent, where the PLM-thiol moves into the solvent. An example of such solvent is chloroform. The organic phase can then be dried, such as by evaporation. The resulting material can be triturated in water for further purification. After trituration in water, a precipitate forms. The precipitate can be collected, washed and dried. A suitable drying condition is at 30 to 70° C. in a vacuum oven for 6-16 h.

In one specific embodiment, the present invention encompasses a process for preparing PLM-thiol precursor of the following Formula A (PLM-thiourea ester)

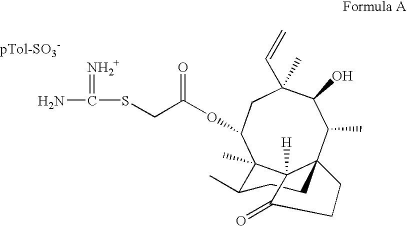

comprising: combining PLM-OLG, organic solvents, and thiourea to obtain a reaction mixture; and maintaining the reaction mixture to obtain PLM-thiourea ester. Preferably, the reaction mixture is maintained for about 2 hours to about 48 hours; more preferably, for about 2 hours to about 36 hours.

PLM-thiourea ester can be recovered from the reaction mixture or can be further converted to PLM-thiol without isolation.

Preferably, the reaction mixture is maintained at a temperature of about 0° C. to about reflux to facilitate the formation of PLM-thiourea ester. More preferably, the reaction mixture is maintained at a temperature of about room temperature to about reflux.

The organic solvent used in the reaction includes but not limited to, C₁-C₈ alcohol, C₃-C₈ ketone, C₃-C₆ ester and C₂-C₈ ethers. Preferably, the solvent includes but not limited to: acetone, MIBK, EtOAc, and methyl tert-butyl ether (MTBE). Most preferably, the solvent is acetone.

In one specific embodiment, the present invention encompasses a process for preparing PLM-thiol precursor of the following Formula A′ (PLM-SAc):

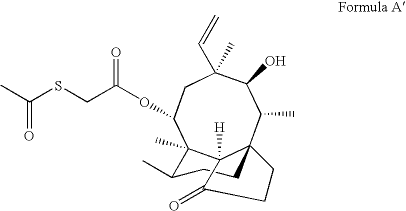

comprising combining PLM-OLG, organic solvents, and thioacetic acid or salts thereof to obtain a reaction mixture; and maintaining the reaction mixture of time to obtain PLM-SAc.

Preferably, the reaction mixture is maintained for about 2 hours to about 48 hours; more preferably, for about 2 hours to about 36 hours.

PLM-SAc can be recovered from the reaction mixture or can be further converted to PLM-thiol without isolation.

Preferably, the reaction mixture is maintained at a temperature of about 0° C. to about reflux to facilitate the formation of PLM-SAc; more preferably, at a temperature of about 15° C. to about 50° C.; and most preferably, at a temperature of about room temperature to about 45° C.

The organic solvent used in the reaction includes but not limited to, C₃-C₈ ketone, C₃-C₆ ester, C₆-C₉ aromatic hydrocarbon and C₂-C₈ ethers. More preferably, the solvent includes but not limited to: THF, MIBK, Acetone, EtOAc, toluene, and methyl tert-butyl ether (MTBE).

Typically, when thioacetic acid is used, a base is added to the reaction. Preferably the base is selected from the group of amines. More preferably, the base is Et₃N.

The recovery of the PLM-thiol precursor such as PLM-thiourea ester or PLM-SAc can be done by different methods, for example, by extraction and evaporation or purification in other solvent system.

The present invention further provides a process for preparing PLM-thiol comprising: combining PLM-thiourea ester or PLM-SAc, an organic solvent, optionally in mixture with water, and a base to obtain a reaction mixture; and maintaining the reaction mixture to obtain PLM-thiol.

Preferably, the reaction mixture is maintained for about 2 hours to about 48 hours; more preferably, for about 2 hours to about 36 hours.

Preferably, the reaction mixture is maintained at a temperature of about 0° C. to about reflux to facilitate the formation of PLM-thiol; more preferably, at a temperature of about 15° C. to about 50° C.; and most preferably, at a temperature of about 25° C. to about 40° C.

The organic solvent used in the reaction includes but not limited to: C₁-C₈ alcohol, C₆-C₉ aromatic, C₃-C₈ ketone, and C₂-C₈ ethers. When the PLM-thiol precursor is PLM-thiourea ester, more preferably, the solvent is ethanol. When the PLM-thiol precursor is PLM-SAc, more preferably, the solvent is selected from the group consisting of: THF, toluene and methyl tert-butyl ether (MTBE).

The base used in the reaction includes but not limited to: sodium thio-sulfite, amines, poly ethylene amines, and alkaline hydroxides. Preferably the base is sodium metabisulfite for the preparation of PLM-thiourea ester or ethylenediamine for the preparation of PLM-SAc).

Preferably the leaving group on the PLM-OLG is mesylate when preparing PLM-SAc, or tosylate when preparing PLM-thiourea ester.

The pleuromutilin derivative of the following Formula II (PLM-OLG)

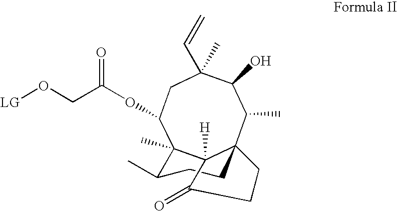

can be prepared by any method know in the art. For Example according to the procedure described in WO20090075776 application, PLM-OLG can be prepared by a process comprising: providing a mixture of pleuromutilin (PLM) of the following Formula I,

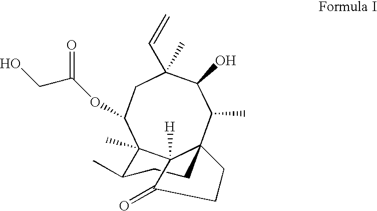

an organic solvent, a base, and a reagent capable of replacing the hydroxyl group in the β position relative to the carbonyl group of the pleuromutilin with a leaving group (“LG”); and maintaining the combination to obtain the pleuromutilin derivative, PLM-OLG.

Any reagent capable of replacing the hydroxyl group in the β position relative to the carbonyl group of the pleuromutilin with a leaving group may be used in the above process. Examples of suitable reagents include, but are not limited to, methane sulphonyl chloride, p-toluenesulfonyl chloride (“TsCl”), thionyl chloride (SOCl₂), thionyl bromide (“SOBr₂”), 4-nitrobenzenesulfonyl chloride (“nosyl chloride;” “NsCl”), benzenesulfonyl chloride, acetyl chloride (“AcCl”), and acetic anhydride (“Ac₂O”).

The organic solvent used in the reaction includes but not limited to, C₃-C₈ ketone, C₃-C₆ ester, C₆-C₉ aromatic hydrocarbon and C₂-C₈ ethers. More preferably the solvent is methyl isobutyl ketone, acetone, ethyl acetate, isobutyl acetate, and toluene.

The base can be alkaline hydroxide or amine The amine can be of the formula of N[R]₃, wherein each R is independently selected from C₁ to C₇ alkyl chain or hydrogen. The amine can be secondary or tertiary amine, and can be selected from a group consisting of triethyl amine, diisopropyl amine, and tributyl amine Most preferably, the alkaline hydroxide is NaOH and the amine is triethylamine.

Typically, the combination is maintained for a period of time and at a temperature sufficient to obtain the pleuromutilin derivative. Preferably, the combination is maintained for about 0.5 to about 24 hours. Preferably, the combination is maintained at a temperature of about 0° C. to about reflux to facilitate the formation of formula II.

The pleuromutilin derivative thus obtained may optionally be further purified by recovered from the reaction mixture by different methods, for example by neutralization, extraction and evaporation.

In another embodiment, the invention encompasses a process for preparing Retapamulin comprising: combining PLM-thiol, organic solvent, base, and tropine-OLG to obtain a reaction mixture; and maintaining the reaction mixture to obtain the Retapamulin.

Preferably, the reaction mixture is maintained for about 16 hours to about 48 hours; more preferably, for about 24 hours to about 36 hours.

Preferably, the reaction mixture is maintained at a temperature of about 0° C. to about reflux to facilitate the formation of Retapamulin; more preferably, at a temperature of about 15° C. to about 50° C.; and most preferably, at a temperature of about 25° C. to about 35° C.

Typically, an antioxidant is introduced into the reaction mixture. Examples of antioxidants can be butylated hydroxytoluene (BHT), butylated hydroxyanisole (BHA).

The organic solvent used in the reaction is preferably a dry solvent. A solvent suitable for using in the process can be selected from the list consisting of: C₂-C₈ ethers, DMF, acetonitrile, C₆-C₈ aromatic hydrocarbon, DMA and N-methyl-2-pyrrolidone (NMP). More preferably, the solvent is selected from the group consisting of: DMF, THF, cyclopentyl methyl ether (CPME), DMA and Toluene.

The base used in the reaction includes but not limited to: Sodium hydride, lithium hydride, sodium tert butoxide, alkaline hydroxides, lithium hexamethyldisilazide (LiHMDS), and amines such as: 1,8-diazabicyclo[5.4.0]undec-7-ene (DBU), 1,4-diazabicyclo[2.2.2]octane (DABCO), 4-dimethylaminopyridine (DMAP), ethylenediamine, and, 2,6-dimethylpyridine(2,6-lutidine). Preferably the base is selected from the group consisting of: Sodium hydride, sodium tert butoxide, and ethylenediamine.

In the process of the invention for obtaining Retapamulin, the isolation of the PLM-thiol is only optional.

In one embodiment, the invention encompasses a one pot reaction for obtaining Retapamulin comprising: converting PLM-OLG or PLM-thiol precursor to PLM-thiol; combining the PLM-thiol with tropine-OLG, base, and organic solvent to obtain a reaction mixture; and maintaining the reaction mixture to obtain the Retapamulin.

Conversion of the PLM-thiol precursor to PLM-thiol can be done as described above.

Preferably, the reaction mixture is maintained for about 16 hours to about 48 hours; more preferably, for about 24 hours to about 36 hours.

Preferably, the reaction mixture is maintained at a temperature of about 15° C. to about 50° C.; more preferably, at a temperature of about 25° C. to about 35° C.

The base used in the reaction is selected from poly ethylene amine. Preferably, the base is ethylenediamine.

Preferably, the PLM-thiol precursor is PLM-SAc or PLM-thiourea ester.

Preferably, the tropine-OLG is tropine-mesylate.

The organic solvent used in the reaction includes but not limited to: C₆-C₉ aromatic hydrocarbon, and C₂-C₈ ethers. More preferably, the organic solvent is selected from the group consisting of THF, and toluene or a mixture thereof. Most preferably, the organic solvent is toluene.

Optionally, an antioxidant, is also introduces into the reaction mixture. Preferably the antioxidant is butylated hydroxytoluene (BHT).

In another embodiment, the present invention provides a one pot reaction for obtaining Retapamulin comprising:

a) converting Tropine to Tropine-OLG in the presence of a solvent selected from the group consisting of: acetone, MIBK and THF;

b) combining Tropine-OLG with a second solvent; and

c) combining the mixture of Tropine-OLG and the second solvent with PLM-thiol precursor or PLM-thiol and a base to obtain a reaction mixture; and

d) maintaining the reaction mixture to obtain the Retapamulin.

Preferably, the reaction mixture is maintained for about 16 hours to about 48 hours; more preferably, for about 24 hours to about 36 hours.

Preferably, the temperature in step a) is about −10° C. to about room temperature; more preferably, about −5° C. to about 10° C.; and most preferably, about 0° C.

Preferably, the reaction mixture in step c) is maintained at a temperature of about 15° C. to about 50° C.; more preferably, at a temperature of about 25° C. to about 35° C.

The base used in the reaction is selected from poly ethylene amine. Preferably, the base is ethylenediamine.

Preferably, the second solvent is selected from C₆-C₈ aromatic hydrocarbon and C₂-C₈ ethers. More preferably, the second solvent is toluene.

Preferably, the PLM-thiol precursor is PLM-SAc.

Preferably, the tropine-OLG is tropine-mesylate.

Exchange of the solvent preferably occurs when introducing the second organic solvent.

In one specific example, following introducing toluene to the Tropine-OLG, acetone is distilled out from the reaction mixture. PLM-thiol precursor or PLM-thiol is then added to the reaction mixture.

The PLM-thiol can be prepared according the above processes.

In one embodiment, the present invention provides a one pot process for the preparation of Retapamulin comprising:

a) in a first vessel, converting PLM to PLM-OLG and further converting the PLM-OLG to PLM-thiol precursor or to PLM-thiol;

b) in a second vessel, converting Tropine to Tropine-OLG in a first solvent that is acetone;

c) combining the reaction mixture of step b) with a second solvent; and

d) combining the PLM-thiol precursor or PLM-thiol with tropine-OLG, and a base to obtain a reaction mixture; and

e) maintaining the reaction mixture to obtain the Retapamulin.

Preferably, the solvent in step a) and c) is C₆-C₈ aromatic hydrocarbon or C₂-C₈ ether. Most preferably, the solvent in step a) and c) is toluene.

Preferably, exchange of the acetone solvent occurs in step c).

Preferably, the PLM-thiol precursor is PLM-SAc.

Preferably, the tropine-OLG is tropine-mesylate.

The base used in the reaction is selected from poly ethylene amine. Preferably, the base is ethylenediamine.

Preferably, the reaction mixture is maintained for about 16 hours to about 48 hours; more preferably, for about 24 hours to about 36 hours.

Preferably, the temperature in step b) is about −10° C. to about room temperature; more preferably, about −5° C. to about 10° C.; and most preferably, about 0° C.

Preferably, the temperature in step d) is about 15° C. to about 50° C.; more preferably, at a temperature about 25° C. to about 35° C.

The recovery of the Retapamulin can be done by different methods, for example by extraction, evaporation, precipitation or purification in other solvent system.

The tropine-OLG of the following Formula B

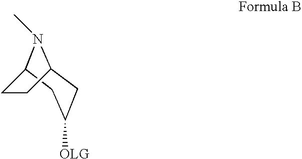

used in the condensation process above for obtaining Retapamulin can be obtained in any method know in the art. For example, tropine-OLG can be prepared in a process comprising: replacing the hydroxyl group of the tropine with a good leaving group.

Preferably the hydroxyl group of the tropine is in the axial position.

Preferably, the leaving group of the tropine mesylate is in the axial position.

Any reagent capable of replacing the hydroxyl group with a leaving group may be used in the above process. Examples of suitable reagents include, but are not limited to, methane sulphonyl chloride, p-toluenesulfonyl chloride (“TsCl”), thionyl chloride (SOCl₂), thionyl bromide (“SOBr₂”), 4-nitrobenzenesulfonyl chloride (“nosyl chloride;” “NsCl”), benzenesulfonyl chloride, acetyl chloride (“AcCl”) and acetic anhydride (“Ac₂O”). Most preferably, the reagent is methane sulfonyl chloride.

Typically, replacing of the hydroxyl group is carried out in the presence of an organic base such as triethyl amine and tributyl amine, and an organic solvent selected from a group consisting of C₂-C₆ linear or branched ketones, C₂-C₈ linear or branched esters and ethers. More preferably the base is triethyl amine and the organic solvent is THF, acetone, MIBK or isobutyl acetate.

The reaction mixture is maintained for a sufficient period of time to obtain tropine-OLG at a temperature of about −10° C. to about reflux to facilitate the formation of the tropine-OLG.

Preferably, the tropine-OLG used in the reaction is tropine mesylate, of the following Formula C.

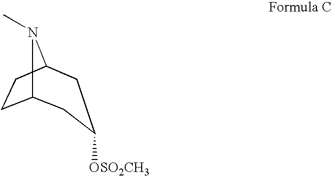

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

The following abbreviations are used throughout the present disclosure with respect to chemical terminology:
BHA Butylated hydroxyanisole
BHT Butylated hydroxytoluene
DMF Dimethylformamide
Et₃N Triethylamine
EtOAc Ethyl acetate
MIBK Methyl isobutyl ketone
MTBE Methyl tert-butyl ether
NMP N-methyl-2-pyrrolidone
PLM Pleoromutilin
THF Tetrahydrofuran
DMA Dimethylacetamide
Tropine-Ms Tropine mesylate

### Example 1

### Preparation of PLM-Tosylate

A 1 L round bottom flask was charged with Pleuromutilin (50 g, 0.1316 mole), tosyl-Cl (38 g, 0.2 mole), methyl tert butyl ether (135 ml) and water (26.5 ml). A solution of NaOH −10N (33 ml) was added drop-wise. The mixture was heated to reflux for 2.5 hours and then cooled to room temperature. Ice water (250 ml) and chloroform (250 ml) were added. The phases were separated. The organic phase was washed with water, with H₂SO₄-3N, with water and then with saturated NaHCO₃. The organic phase was dried on Na₂SO₄ anhydrous and then evaporated to dryness to give a white product (66.5 g). The white product was vigorously stirred with n-hexane (1150 ml) at room temperature for over night. The precipitant was filtrated under reduced pressure and dried at 35° C. in a vacuum oven over night to give PLM-tosylate (62.8 g, 99% purity)

### Example 2

### Preparation of PLM-Thiourea Ester

A 1 L three necked round bottom flask was charged with PLM-tosylate (52.5 g), thiourea 99% (Aldrich, 7.5 g, 1 eq.) and acetone (130 ml, 2.5 vol.). The reaction mixture was heated to reflux and stirred for 24 hours. The solution was evaporated and the residue was vigorously stirred with n-hexane (650 ml) at room temperature for 5 hours. The precipitant was filtrated under reduced pressure and dried at 35° C. in a vacuum oven over night to give PLM-thiourea-ester (60 g).

### Example 3

### Preparation of PLM-Thiol

A 1 L round bottom flask was charged with PLM-thiourea-ester (59 g), abs ethanol (95 ml) and water (171 ml). The slurry was heated to reflux to obtain a solution. A solution of Na₂S₂O₅ (22 g) in water (120 ml) was added drop-wise, the obtained slurry was stirred at reflux for 1 hour and then cooled to room temperature. The slurry was extracted with chloroform and the phases were separated. The organic phase was evaporated to have sticky product (37 g) which was stirred vigorously in water (370 ml) at room temperature for over night. The product was precipitated, filtered under reduced pressure, washed with water and dried at 35° C. in a vacuum oven over night to give 35.3 g of PLM-thiol (98.3% purity).

### Example 4

### Coupling Reaction—General Procedure:

A 3 neck-flask (50 ml) equipped with a stirring bar and a reflux condenser is dried under N₂. Dry solvent (10 vol) and PLM-thiol (1 eq.) were charged. The reaction mixture was heated to 35° C. Base (1.14 eq.) was added, the reaction mixture was stirred for 0.5 h. Tropine-mesylate (1.14 eq.) was added and the reaction mixture stirred over-night.

### Example 5

### Preparation of Tropine Mesylate

To a 500 ml round bottom flask with mechanical stirrer, and nitrogen inlet charge, tropine (10 g, 70.81 mmol), MIBK (200 ml) and triethylamine (14.31 g, 141.63 mmol) were charged. Clear solution was obtained. The reaction was cooled to −10° C., methane sulfonyl chloride (18.49 g, 164.55 mmol) was added dropwised while still cooling the reaction to −10° C. Solid started to precipitate. The reaction maintained to reach room temperature and the reaction was stirred at room temperature over night. The reaction was monitored by HPLC and complete conversion was obtained (the product was not isolated).

### Example 6

### Preparation of PLM-Mesylate in Toluene

To a solution of Toluene (300 ml), Tri ethyl amine (28.6 ml) and Pleuromutilin (50 g), Methyl sulfonyl chloride (15.3 ml) was added dropwise at room temperature. The solution became white massive slurry. The reaction mixture was stirred for 2 h at room temperature and then vacuum filtration was done. The reaction was monitored by HPLC, full conversion was obtained.

### Example 7

### Preparation of PLM-SAc

Into 250 ml round bottom flask with magnetic stirrer, Pleuromutilin-mesylate (7 g, 15.3 mmol) and potassium thioacetate (1.82 gr, 1.04 eq) and THF (50 ml) were charged. The reaction was stirred for 4 h at room temperature solution (gel). The reaction was evaporated almost to dryness and EtOAc (70 ml) was added and extracted with water. The solution was dried (MgSO₄) filtrated and evaporated. PLM-SAc was obtained in quantitative yield.

### Example 8

### Preparation of PLM-SAc

Into 250 ml round bottom flask with magnetic stirrer, Pleuromutilin-mesylate (5 g, 15.3 mmol) and MIBK (100 ml) and potassium thioacetate (1.3 gr) were charged. The reaction was stirred for 2 h at room temperature solution (gel). The reaction was extracted with water. PLM-SAc was obtained in quantitative yield.

### Example 9

### Preparation of PLM-SAc

Into 1 L round bottom flask with magnetic stirrer, Pleuromutilin-mesylate (20 g), EtOAc (400 ml) and thioacetic acid (3.25 ml) and Et₃N (6.6 ml) were charged. The reaction was stirred for over night at room temperature afterwards heated to 40° C. and stirred for additional 24 h. The reaction was cooled and extracted with water, dried on MgSO₄, and evaporated to dryness. PLM-SAc was obtained in quantitative yield as oil which was slowly crystallized.

### Example 10

### Preparation of PLM-SAc

Into 3 L round bottom flask with mechanical stirrer, PLM-mesylate (200 g), EtOAc (1600 ml), Et₃N (70 ml) and thioacetc acid (33 ml) were charged. The reaction was stirred for 18 h at room temperature as solution. The reaction was washed with water. The solution was dried (Na₂SO₄) filtrated and evaporated. To the obtained oil Cyclohexane (750 ml) was added. After one hour the oil crystallized and stirred as slurry 20 hrs. The product was filtered and washed with Cyclohexane. PLM-SAc was obtained 95% yield.

### Example 11

Preparation of PLM-Thiol from PLM-SAc

Into 250 ml 3 necked round bottom flask with magnetic stirrer, under N₂ bubbling, Pleuromutilin-SAc (4.7 g, 10.8 mmol) and THF (50 ml) were charged. Ethylenediamine was added (2.2 ml, 3 eq), the solution turned yellow and then colorless. The reaction was stirred for 1 h at room temperature solution (gel). The reaction was evaporated almost to dryness and EtOAc (70 ml) was added and extracted with water and brine (2×5 ml). The solution was dried (MgSO₄), filtrated and evaporated. PLM-thiol was obtained.

### Example 12

Preparation of Retapamulin from PLM-SAc in EDA

Into 20 ml vial with magnetic stirrer, Pleuromutilin-SAc (1 g, 2.3 mmol) Ethylenediamine (3 ml) were charged. The reaction was stirred for 1 h at room temperature solution. tropine-Ms (0.5 gr, 1 eq) was added. The reaction was stirred for over night at room temperature solution. No isolation, Retapamulin—37% conversion (by assay).

### Example 13

Preparation of Retapamulin from PLM-SAc in THF

Into 100 ml 3 necked round bottom flask with magnetic stirrer, under N₂ bubbling, Pleuromutilin-SAc (3.3 g, 6.8 mmol) and THF (25 ml) and BHT (30 mg) were charged. Ethylenediamine (7.6 ml, 20 eq) and afterwards tropine-Ms (1.25 gr) were added. The reaction was stirred for 48 h at room temperature solution. No isolation, Retapamulin—54% conversion (by assay).

### Example 14

Preparation of Retapamulin from PLM-SAc in Toluene

Into 100 ml 3 necked round bottom flask with magnetic stirrer, under N₂ bubbling, tropine-Ms (3.5 gr) and toluene (14 ml) and Ethylenediamine (13.1 ml) were charged. PLM-SAc (7.2 gr) was added. The reaction was stirred for 24 h at 35° C. No isolation, Retapamulin—50% conversion (by assay).

### Example 15

Preparation of Retapamulin from PLM-SAc in Toluene (Isolation)

Into 100 ml 3 necked round bottom flask with magnetic stirrer, under N₂ bubbling, PLM-SAc (15 gr) and toluene (45 ml) were charged. Ethylenediamine (13.3 ml) and tropine-Ms (6.25 gr) were added. The reaction was stirred for 24 h at 35° C. No isolation, Retapamulin—48% conversion (by assay). Methanol (20 ml) was added, and the reaction mixture was evaporated. Toluene (60 ml) and water (45 ml) were added and the pH was adjusted to 8 (HCl 2N), the phases were separated. The organic phase was extracted with water (45 ml) and the pH was adjusted to 8. The organic phase was extracted with water and the pH was adjusted to 2. After separation, the pH of the aqueous phase was adjusted to 10 with 2N NaOH. The mixture was stirred at room temperature for 20 hours. The product was vacuum filtered and washed with water. The collected crystals were dried in a 50° C. vacuum oven to yield 31% of isolated Retapamulin.

### Example 16

Preparation of Retapamulin from PLM-SAc in Toluene (Isolation)

Into 100 ml 3 necked round bottom flask with magnetic stirrer, under N₂ bubbling, tropine-Ms (3.5 gr) and toluene (42 ml) and Ethylenediamine (17.5 ml) were charged. PLM-SAc (7.2 gr) was added. The reaction was stirred for 24 h at 35° C.

The reaction mixture was evaporated. Toluene (100 ml) and water (100 ml) were added, the phases were separated. Water (70 ml) were added to the organic phase and the pH was adjusted to 8 (HCl 2N), the phases were separated. Water (70 ml) was added to the organic phase and the pH was adjusted to 8 (HCl 2N). The phases were separated. Water (120 ml) was added to the organic phase water the pH adjusted to 1.5 (HCl 2N), the phases separated. The aqueous phase was evaporated and fresh water (300 ml) was added. The pH was adjusted to 10 with NaOH 2N. The mixture was stirred at room temperature for 20 hours. The product was filtrated under vacuum and washed with water. The solid was dried in 55° C. vacuum oven to yield Retapamulin in Yield 39%.

### Example 17

Preparation of Retapamulin from PLM-SH in THF

Into 250 ml 3 necked round bottom flask with magnetic stirrer, under N₂ bubbling, Pleuromutilin-SH (3 g, 7.6 mmol) and THF (50 ml, 6.6 vol) were charged. Ethylenediamine (2 ml, 4 eq) was added afterwards tropine-Ms (1.66 gr, 1 eq) was added. The reaction was stirred for 48 h at room temperature solution. No isolation, Retapamulin—46% conversion (by assay).

### Example 18

### Preparation of Retapamulin—One Pot Reaction

To a 100 ml reactor occupied with mechanical stirrer, Tropine (5 g, 35.4 mmol), acetone extra dry (75 ml) and Et₃N (7.5 ml) were added.

The reaction was cooled to 0° C. Methane sulfonyl chloride (3.3 ml) was added via syringe pump (1 hour), while keeping the temperature at 0° C. during the addition. When the addition was ended, the reaction was heated to 25° C. and then stirred for additional 2 h.

The slurry was filtered and washed with acetone, the filtrate was returned into a 250 ml round bottom flask. Toluene (40 ml) was added and the solution was evaporated until almost dryness. Toluene (25 ml) and ethylenediamine (28 ml) and PLM-SAc (15 gr) were added under N₂. The mixture was stirred at 35° C. for 24 h.

The two phases in the reaction mixture were separated. To the toluene phase, toluene (75 ml) and water (100 ml) were added, the phases were separated. Water (70 ml) were added to the organic phase and the pH was adjusted to 8 (HCl 2N), the phases were separated. Water (70 ml) was added to the organic phase and the pH was adjusted to 8 (HCl 2N). The phases were separated. Water (120 ml) was added to the organic phase and the pH was adjusted to 1.5 (HCl 2N), the phases separated. The aqueous phase was washed with toluene was added. Water (300 ml) was added to the aqueous phase. The pH was adjusted to 10 with NaOH 2N. The mixture was stirred at room temperature for 6 h. The product was filtrated under vacuum and washed with water. The solid was dried in 55° C. vacuum oven. Retapamulin—4.9 gr, Yield 41%.

### Example 19

### Preparation of Tropine Mesylate

To a 250 ml round bottom flask with mechanical stirrer, tropine (10 g, 70.81 mmol), ethyl acetate (100 ml) and triethylamine (10 g, 99 mmol) were charged. A diluted slurry was obtained. The reaction was cooled to −3° C., methane sulfonyl chloride (10 g, 87 mmol) dissolved in ethyl acetate (10 ml) was added dropwise while still cooling the reaction to −3° C. A solid started to precipitate (massively). The reaction was maintained to reach room temperature and the reaction was stirred at room temperature over night. The reaction was monitored by HPLC, 87% conversion was obtained. The product was not isolated with a 63% yield by assay.

### Example 20

### Preparation of Tropine Mesylate (Tropine-Ms)

To a 250 ml round bottom flask with mechanical stirrer, tropine (10 g, 70.81 mmol), isobutyl acetate (100 ml) and triethylamine (10 g, 99 mmol) were charged. A diluted slurry was obtained. The reaction was cooled to −3° C., Methane sulfonyl chloride (10 g, 87 mmol) dissolved in isobutyl acetate (10 ml) was added dropwise while still cooling the reaction to −3° C. Solid was started to precipitate massively, the reaction was maintained to reach room temperature and the reaction was stirred at room temperature for over night. The reaction was monitored by HPLC, 83% conversion was obtained. The product was not isolated with a 66% yield by assay.

### Example 21

### Preparation of Tropine Mesylate (Tropine-Ms)

To a 150 ml reactor, tropine (10 g, 70.81 mmol), acetone (100 ml) and triethylamine (15 ml, 212 mmol) were charged. The reaction mixture was stirred at room temperature for 20 min, cooled to −5° C. and methane sulfonyl chloride (11.6 g, 101 mmol) was added dropwise via syringe pump. The temperature was raised to 5° C. and the reaction was stirred at 5° C. over night. The reaction was monitored by HPLC; the product was not isolated with a 79% yield by assay.

### Example 22

### Preparation of Tropine Mesylate (Tropine-Ms)

To a 150 ml reactor, tropine (10 g, 70.81 mmol), THF (100 ml) and triethylamine (15 ml, 212 mmol) were charged. The reaction was stirred at room temperature for 30 min, cooled to −5° C. and methane sulfonyl chloride (11.6 g, 101 mmol) was added dropwise via syringe pump. The temperature was raised to 15° C., and the reaction was stirred at 15° C. over night. The reaction was monitored by HPLC; the product was not isolated with a 88% yield by assay.

### Example 23

### Preparation of Tropine Mesylate (Tropine-Ms)

To a 250 ml flask with mechanical stirrer, tropine (10 g, 70.81 mmol), MIBK (100 ml) and triethylamine (15 ml, 212 mmol) were charged. The reaction was stirred and cooled in ice/acetone bath, Methane sulfonyl chloride (11.6 g, 101 mmol) was added dropwise via syringe pump. The temperature was raised to room temperature, and the reaction was stirred at room temperature for over night. The reaction was monitored by HPLC; the product was not isolated with a 84% yield by assay.

1. A process for the preparation of PLM-thiol (formula III): 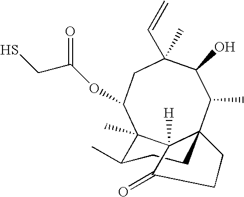
comprising: combining PLM-OLG, an organic solvent, optionally in mixture with water, S-donor and a base to obtain a reaction mixture; and maintaining the reaction mixture to obtain PLM-thiol.

2. The process according to claim 1, wherein the reaction mixture is maintained for about 2 hours to about 48 hours.

3. The process according to claim 1, wherein the reaction mixture is maintained at a temperature of about 0° C. to about reflux.

4. The process according to claim 1, wherein the organic solvent used in the reaction is selected from the group consisting of one or more of: C₆-C₈ aromatic hydrocarbons, C₁-C₈ alcohol, C₃-C₈ ketone, C₃-C₇ esters, C₂-C₈ ethers, and combinations thereof.

5. The process according to claim 4, wherein the organic solvent is selected from the group consisting of: ethanol, THF, MIBK, acetone, EtOAc toluene, methyl tert-butyl ether (MTBE), and combinations thereof.

6. The process according to claim 1, wherein the base is selected from the group consisting of: sodium thio-sulfite, amines, poly ethylene amines, and alkaline hydroxides.

7. The process according to claim 6, wherein the base is sodium metabisulfite or ethylenediamine.

8. The process according to claim 1, wherein the S-donor is selected from the group consisting of: thiourea, thioacetic acid and salts thereof, sodium sulfide, sodium hydrosulfide, sodium xanthate.

9. The process according to claim 8, wherein the S-donor is thiourea or thioacetic acid and its salts.

10. The process according to claims 1, wherein the leaving group on the PLM-OLG is mesylate or tosylate.

11. A process for the preparation of PLM-thiol comprising: combining PLM-thiourea ester or PLM-SAc, an organic solvent, optionally in mixture with water, and a base to obtain a reaction mixture; and maintaining the reaction mixture to obtain PLM-thiol.

12. The process according to claim 11, wherein the reaction mixture is maintained for about 2 hours to about 48 hours.

13. The process according to claim 11, wherein the reaction mixture is maintained at a temperature of about 0° C. to about reflux.

14. The process according to claim 11, wherein the organic solvent is selected from the group consisting of: C₁-C₈ alcohol, C₆-C₈ aromatic hydrocarbon, C₃-C₈ ketone, and C₂-C₈ ethers.

15. The process according to claim 14, wherein the precursor is PLM-thiourea ester, the organic solvent is ethanol

16. The process according to claim 14, wherein the precursor is PLM-SAc, the organic solvent is selected from the group consisting of: THF, toluene and methyl tert-butyl ether (MTBE).

17. The process according to claim 11, wherein the base is selected from the group consisting of: sodium thio-sulfite, amines, poly ethylene amines, and alkaline hydroxides.

18. The process according to claim 15, wherein the base is sodium metabisulfite or ethylnediamine.

19. A process for the preparation of Retapamulin comprising: combining PLM-thiol, organic solvent, base, and tropine-OLG to obtain a reaction mixture; and maintaining the reaction mixture to obtain the Retapamulin.

20. The process according to claim 19, wherein the reaction mixture is maintained for about 16 hours to about 48 hours.

21. The process according to claims 19, wherein the reaction mixture is maintained at a temperature of about 0° C. to about reflux.

22. The process according to claim 19, wherein an antioxidant, selected from the group consisting of: butylated hydroxytoluene (BHT) and butylated hydroxyanisole (BHA), is introduced into the reaction mixture.

23. The process according to claim 19, wherein the organic solvent is selected from the group consisting of: C₂-C₈ ethers, DMF, acetonitrile, C₆-C₉ aromatic hydrocarbons, DMA and N-methyl-2-pyrrolidone (NMP).

24. The process according to claim 23, wherein the organic solvent is selected from the group consisting of: DMF, THF, and cyclopentyl methyl ether (CPME), DMA and Toluene.

25. The process according to claim 19, wherein the base is selected from the group consisting of: Sodium hydride, lithium hydride, sodium tert butoxide, alkaline hydroxides, lithium hexamethyldisilazide (LiHMDS), and amines such as: 1,8-diazabicyclo[5.4.0]undec-7-ene (DBU), 1,4-diazabicyclo[2.2.2]octane (DABCO), 4-dimethylaminopyridine (DMAP), ethylene diamine, and, 2,6-dimethylpyridine(2,6-lutidine).

26. The process according to claim 25, wherein the base is selected from the group consisting of: Sodium hydride, sodium tert butoxide, and ethylenediamine.

27. A one pot reaction process for the preparation of Retapamulin comprising: converting PLM-OLG or PLM-thiol precursor to PLM-thiol; combining the PLM-thiol with tropine-OLG, base, and organic solvent to obtain a reaction mixture; and maintaining the reaction mixture to obtain the Retapamulin.

28. The process according to claim 27, wherein the reaction mixture is maintained for about 10 hours to about 48 hours.

29. The process according to claim 27, wherein the reaction mixture is maintained at a temperature of about 15° C. to about 50° C.

30. The process according to claim 27, wherein the base is selected from poly ethylene amine.

31. The process according to claim 30, wherein the base is ethylenediamine.

32. The process according to claim 26, wherein the PLM-thiol precursor is PLM-SAc

33. The process according to claim 27, wherein the tropine-OLG is tropine-mesylate.

34. The process according to claim 27, wherein the organic solvent is selected from the group consisting of: C₆-C₈ aromatic hydrocarbon and C₂-C₈ ethers.

35. The process according to claim 34, wherein the organic solvent is selected from the group consisting of: THF, toluene and a mixture thereof.

36. The process according to claim 35, wherein the organic solvent is toluene.

37. The process according to claim 27, wherein an antioxidant is introduced into the reaction mixture.

38. A one pot reaction process for the preparation of Retapamulin comprising:
a. converting Tropine to Tropine-OLG in the presence of a first solvent selected from the group consisting of: acetone, MIBK and THF;
b. combining Tropine-OLG with a second solvent;
c. combining the mixture of Tropine-OLG and the second solvent with PLM-thiol precursor or PLM-thiol and a base to obtain a reaction mixture; and
d. maintaining the reaction mixture to obtain the Retapamulin.

39. The process according to claim 38, wherein the reaction mixture is maintained for about 16 hours to about 48 hours.

40. The process according to claim 38, wherein the temperature in step a) is about −10° C. to about room temperature.

41. The process according to claim 40, wherein the temperature in step c) is about 15° C. to about 50° C.

42. The process according to claim 38, wherein the base is selected from poly ethylene amine.

43. The process according to claim 42, wherein the base is ethylenediamine.

44. The process according to claim 38, wherein the second solvent is selected from the group of C₆-C₈ aromatic hydrocarbons and C₂-C₈ ethers.

45. The process according to claim 44, wherein the second solvent is toluene.

46. The process according to claim 38, wherein the PLM-thiol precursor is PLM-SAc.

47. The process according to claim 38, wherein the tropine-OLG is tropine-mesylate.

48. The process according to claim 38, wherein exchange of the solvent occurs when introducing the second organic solvent.

49. A one pot reaction process for the preparation of Retapamulin comprising:
a) in a first vessel, converting PLM to PLM-OLG and further converting the PLM-OLG to PLM-thiol precursor or to PLM-thiol;
b) in a second vessel, converting Tropine to Tropine-OLG in a first solvent that is acetone;
c) combining the reaction mixture of step b) with a second solvent; and
d) combining the PLM-thiol precursor or PLM-thiol with tropine-OLG, and a base to obtain a reaction mixture; and
e) maintaining the reaction mixture to obtain the Retapamulin.

50. The process according to claim 49, wherein the solvent in step a) and c) is C₆-C₈ aromatic hydrocarbon or C₂-C₈ ether.

51. The process according to claim 50, wherein the solvent in step a) and c) is toluene.

52. The process according to claim 49, wherein exchange of the acetone solvent occurs in step c).

53. The process according to claim 49, wherein the base is selected from poly ethylene amine.

54. The process according to claim 53, wherein the base is ethylenediamine.

55. The process according to claim 49, wherein the PLM-thiol precursor is PLM-SAc.

56. The process according to claim 49, wherein the tropine-OLG is tropine-mesylate.

57. The process according to claim 49, wherein the reaction mixture is maintained for about 16 hours to about 48 hours.

58. The process according to claim 49, wherein the temperature in step b) is about -10° C. to about room temperature.

59. The process according to claim 58, wherein the temperature in step d) is about 15° C. to about 50° C.
